(19) United States
(12) Patent Application Publication
(10) Pub. No.: US 2019/0069528 A1
(43) Pub. Date: Mar. 7, 2019

(54) ENGINEERED HUMAN HOOKWORMS AS A NOVEL BIODELIVERY SYSTEM FOR VACCINES AND BIOLOGICALS

(71) Applicant: The George Washington University, Washington, DC (US)

(72) Inventors: John M. HAWDON, Bowie, MD (US); Douglas NIXON, Washington, DC (US); Suhao HAN, Vienna, VA (US); Claudia BROCKMEYER, Wantage (GB); Jeffrey BETHONY, Washington, DC (US); Ramesh RATNAPPAN, Derwood, MD (US); David DIEMERT, Washington, DC (US)

(21) Appl. No.: 16/087,196

(22) PCT Filed: Mar. 21, 2017
(86) PCT No.: PCT/US17/23419

§ 371 Date: Sep. 21, 2018

Related U.S. Application Data
(60) Provisional application No. 62/311,185, filed on Mar. 21, 2016.
(60) Provisional application No. 62/450,453, filed on Jan. 25, 2017.

(51) Int. Cl. A01K 67/033; A61K 39/12; C12N 15/85; A61K 9/00; A61P 31/18

(52) U.S. Cl. CPC A01K 67/0336; A61K 39/12; C12N 15/8509; C12N 2740/16021; A61K 9/0014; A61P 31/18; A61K 9/0053

(57) ABSTRACT

The present disclosure generally relates to genetic methods of manipulating helminths, e.g., hookworms, to act as a biological delivery vehicle for therapeutic polypeptides in mammals. Furthermore, the disclosure is drawn to compositions comprising genetically modified hookworms and methods of use, including the administration of the helminths to one or more mammals to provide a continuous supply of a synthetic or modified polypeptide (e.g., HIV neutralizing antibodies) which may mitigate infection and/or infection intensity, otherwise resulting in an increase in a desirable phenotypic trait.

### CROSS-REFERENCE TO RELATED APPLICATIONS

This application claims the benefit of priority to U.S. Provisional Application No. 62/311,185, filed Mar. 21, 2016; and U.S. Provisional Application No. 62/450,453 filed Jan. 25, 2017; each of which is herein incorporated by reference in its entirety.

### STATEMENT REGARDING FEDERALLY SPONSORED RESEARCH OR DEVELOPMENT

This invention was made with government support under R21 A1101369 awarded by the NIH; and under AI117970 from the District of Columbia Center for AIDS Research and the NIH. The U.S. Government has certain rights in the invention.

### STATEMENT REGARDING SEQUENCE LISTING

The sequence listing associated with this application is provided in text format in lieu of a paper copy, and is hereby incorporated by reference into the specification. The name of the text file containing the sequence listing is GWUV_010_02WO_ST25.txt. The text filed is 4 kb and was created on Mar. 20, 2017, and is being submitted electronically via EFS-Web.

### FIELD OF THE INVENTION

The present disclosure generally relates to genetic methods of manipulating hookworms to act as a biological delivery vehicle for therapeutic polypeptides in mammals, e.g., the continuous delivery of bovine growth hormone in cattle or the continuous delivery of HIV neutralizing antibodies.

### BACKGROUND

The delivery of biological molecules into mammals has a global application across a wide swath of medical categories, including the prophylaxis and treatment of infectious diseases, metabolic diseases, genetic diseases, and/or autoimmune diseases. Despite significant advances in the fields of disease treatment, management, and prevention, a considerable barrier exists in the ability to easily and painlessly deliver a constant stream of bioactive molecules to a mammal in the absence of repeated parenteral treatment or repeated oral administration. Biological molecules can include anti-hemophilic factors, synthetic biological molecules, antibodies, antitoxins, hormones, chemokines, cytokines, defensins, antigens, etc. For example, HIV is known to be susceptible to neutralizing antibodies and the ability to easily deliver a constant stream of HIV-specific neutralizing antibodies into the human circulatory system would prove to be a boon for HIV prophylaxis and management.

One approach for delivering such biological molecules is the repeated parenteral delivery on a daily or weekly basis. However significant problems can arise through the insertion of a catheter or port, or even repeated use of syringes. While oral delivery of therapeutics may exhibit an ease of use, many therapeutics are not capable of maintaining biological activity after exposure to the upper gastrointestinal tract. Another approach for delivery is the use of gene therapy to insert one or more genes into the host or patient; however, gene therapy applications are associated with problems known to arise due to the random nature of gene insertion that may cause genetic abnormalities such as cancer.

There is clearly a need for improved methods and compositions for delivering biological molecules over sustained periods of time while mitigating the problems associated with treatments currently used in the art.

### SUMMARY OF THE DISCLOSURE

In some aspects, a transgenic helminth comprises cells containing a polynucleotide sequence comprising one or more control sequences operably linked to at least one heterologous nucleic acid sequence, wherein the at least one heterologous nucleic acid sequence encodes a vaccine antigen and/or a therapeutic polypeptide. In further aspects, the helminth is a trematode, cestode, or nematode. In further aspects, the helminth is a nematode selected from the genera consisting of: Necator, Ancylostoma, Agriostomum, Bunostomum, Cyclodontostonium, Galonchus, Monodontus, Uncinaria, Enterobius, Trichuris, Capillostrongyloides, Liniscus, Orthominx, Pearsonema, Sclerotrichum, Strongyloides, and Tenoranema.

In some aspects, the transgenic helminth is a hookworm. In some aspects, the hookworm is selected from Ancylostonia braziliense, Ancylostoma caninum, Ancylostoma ceylanicum, Ancylostoma duodenale, Ancylostoma pluridentatum, Ancylostoma tubaeforme, Necator amnericanus, and Uncinaria stenocephala.

In some aspects, the vaccine antigen is an HIV polypeptide, which may be an envelope V2 region polypeptide. In some aspects, the therapeutic polypeptide is selected from insulin, gamma-interferon, beta-interferon, Factor VIII, Factor IX, tissue plasminogen activator, human growth hormone, bovine growth hormone, and a neutralizing antibody. In some aspects, the therapeutic polypeptide is a neutralizing antibody, which neutralizes viral envelope proteins. In further aspects, the therapeutic polypeptide as a neutralizing antibody of VRC01.

In some aspects, the at least one heterologous nucleic acid sequence further encodes an adjuvant. In further aspects, the adjuvant is selected from flagellin, Escherichia coli heat labile toxin, cholera toxin, an AB5 toxin, a viral coat protein, a chemokine, a cytokine, and a defensin.

In some aspects, the one or more control sequences of the transgenic hookworm is selected from the group consisting of a promoter, a terminator, a secretion signal, an enhancer, and an operator. In further aspects, the one or more control sequences is a hookworm promoter. In further aspects, the hookworm promoter is selected from asp-1, asp-2, asp-3, asp-4, asp-5, snr-3, lpp-1, tbg-1, myo-2, myo-3, ges-1, eft-3, ama-1, daf-11, daf-16, daf-21, daf-2, let-858, unc-119, vit-2, sur-5, hlh-13, pie-1, and spe-11. In some aspects, the hookworm promoter is a stage-specific promoter that is parasitic L3 stage-specific, L4 stage-specific, or adult stage-specific.

In some aspects, the at least one heterologous nucleic acid sequence encodes an HIV envelope V2 region and an AB5 toxin.

In some aspects, the disclosure is drawn to a method of preparing a transgenic helminth, the method comprising introducing into cells of a helminth a polynucleotide comprising one or more control sequences operably linked to at least one heterologous nucleic acid sequences, wherein the at least one heterologous nucleic acid sequence encodes a vaccine antigen and/or a therapeutic polypeptide. In further aspects, the helminth is a trematode, cestode, or nematode. In further aspects, the helminth is a nematode selected from the genera consisting of: Necator, Ancylostoma, Agriostomum, Bunostomum, Cyclodontostomum, Galonchus, Monodontus, Uncinaria, Enterobius, Trichuris, Capillostrongyloides, Liniscus, Orthominx, Pearsonema, Sclerotrichum, Strongyloides, and Tenoranema.

In some aspects, the transgenic helminth is a hookworm. In some aspects, the hookworm is selected from Ancylostoma braziliense, Ancylostoma caninum, Ancylostoma ceylanicum, Ancylostoma duodenale, Ancylostoma pluridentatum, Ancylostoma tubaeforme, Necator amnericanus, and Uncinaria stenocephala.

In some aspects, the method comprises the introduction of a polynucleotide into cells of the helminth through biolistic bombardment or viral transfection. In some aspects, the vaccine antigen is an HIV polypeptide. In further aspects, the HIV polypeptide is an envelope V2 region polypeptide. In some aspects, the therapeutic polypeptide is selected from insulin, gamma-interferon, beta-interferon, Factor VIII, Factor IX, tissue plasminogen activator, human growth hormone, bovine growth hormone, and a neutralizing antibody. In some aspects, the therapeutic polypeptide is a neutralizing antibody, which neutralizes viral envelope proteins. In further aspects, the neutralizing antibody neutralizes HIV envelope proteins. In further aspects, the therapeutic polypeptide is a neutralizing antibody of VRC01.

In some aspects, the at least one heterologous nucleic acid sequence further encodes an adjuvant. In further aspects, the adjuvant is selected from flagellin, Escherichia coli heat labile toxin, cholera toxin, an AB5 toxin, a viral coat protein, a chemokine, a cytokine, and a defensin.

In some aspects, the one or more control sequences are selected from a promoter, a terminator, a secretion signal, an enhancer, and an operator. In some aspects, the one or more control sequences is a hookworm promoter. In further aspects, the hookworm promoter is a stage-specific promoter that is parasitic L3 stage-specific, L4 stage-specific, or adult stage-specific. In further aspects, the hookworm promoter is selected from asp-1, asp-2, asp-3, asp-4, asp-5, snr-3, lpp-1, tbg-1, myo-2, myo-3, ges-1, eft-3, ama-1, daf-1, daf-16, daf-21, daf-2, let-858, unc-119, vit-2, sur-5, hlh-13, pie-1, and spe-11.

In some aspects, the at least one heterologous nucleic acid sequence encodes an HIV envelope V2 region and an AB5 toxin.

In some aspects, the method of introducing a polynucleotide into cells of a hookworm comprise viral transfection utilizing a lentivirus vector to introduce the polynucleotide, wherein the lentivirus vector has been pseudotyped with a VSV-G envelope protein. In some aspects, the method of introducing a polynucleotide into cells of a hookworm comprises a viral transfection utilizing a retrovirus vector to introduce the polynucleotide, wherein the retrovirus vector has been pseudotyped with a VSV-G envelope protein.

In some aspects, the disclosure is drawn the a method of delivering one or more polypeptides to a mammalian circulatory system or intestinal tract, the method comprising delivering via either an oral or percutaneous route, a composition comprising a transgenic helminth comprising cells containing a polynucleotide comprising one or more control sequences operably linked to at least one heterologous nucleic acid sequence, wherein the at least one heterologous nucleic acid sequence encodes a vaccine antigen and/or a therapeutic polypeptide.

In further aspects, the helminth is a trematode, cestode, or nematode. In further aspects, the helminth is a nematode selected from the genera consisting of: Necator, Ancylostoma, Agriostomum, Bunostomum, Cyclodontostomum, Galonchus, Monodontus, Uncinaria, Enterobius, Trichuris, Capillostrongyloides, Liniscus, Orthominx, Pearsonema, Sclerotrichum, Strongyloides, and Tenoranema.

In some aspects, the transgenic helminth is a hookworm. In some aspects, the hookworm is selected from Ancylostoma braziliense, Ancylostoma caninum, Ancylostoma ceylanicunm, Ancylostoma duodenale, Ancylostoma pluridentatum, Ancylostoma tubaeforme, Necator americanus, and Uncinaria stenocephala.

In some aspects, the method comprises the introduction of a polynucleotide into cells of the helminth through biolistic bombardment or viral transfection. In some aspects, the vaccine antigen is an HIV polypeptide. In further aspects, the HIV polypeptide is an envelope V2 region polypeptide. In some aspects, the therapeutic polypeptide is selected from insulin, gamma-interferon, beta-interferon, Factor VIII, Factor IX, tissue plasminogen activator, human growth hormone, bovine growth hormone, and a neutralizing antibody. In some aspects, the therapeutic polypeptide is a neutralizing antibody, which neutralizes viral envelope proteins. In further aspects, the neutralizing antibody neutralizes HIV envelope proteins. In further aspects, the therapeutic polypeptide is a neutralizing antibody of VRC01.

In some aspects, the at least one heterologous nucleic acid sequence further encodes an adjuvant. In further aspects, the adjuvant is selected from flagellin, Escherichia coli heat labile toxin, cholera toxin, an AB5 toxin, a viral coat protein, a chemokine, a cytokine, and a defensin.

In some aspects, the one or more control sequences are selected from a promoter, a terminator, a secretion signal, an enhancer, and an operator. In some aspects, the one or more control sequences is a hookworm promoter. In further aspects, the hookworm promoter is a stage-specific promoter that is parasitic L3 stage-specific, L4 stage-specific, or adult stage-specific. In further aspects, the hookworm promoter is selected from asp-1, asp-2, asp-3, asp-4, asp-5, snr-3, lpp-1, tbg-1, myo-2, myo-3, ges-1, eft-3, ama-1, daf-11, daf-16, daf-21, daf-2, let-858, unc-119, vit-2, sur-5, hlh-13, pie-1, and spe-11.

In some aspects, the at least one heterologous nucleic acid sequence encodes an HIV envelope V2 region and an AB5 toxin.

In some aspects, the disclosure is drawn to a method of treating an HIV-infected patient, the method comprising orally or percutaneous administration of the transgenic hookworm to a patient in need thereof. In further aspects, about one to three months post-hookworm introduction, the patient exhibits a decreased HIV titer, as compared to the HIV titer immediately before hookworm introduction. In further aspects, the neutralizing antibody is VRC01.

In some aspects, the disclosure is drawn to a method of HIV prophylaxis in a patient, the method comprising orally or percutaneously administering a transgenic hookworm of the present disclosure to a patient in need thereof. In further aspects, the patient is protected from HIV infection at a greater occurrence than a patient not having been administered the transgenic hookworm. In further aspects, the HIV polypeptide is an envelope V2 region polypeptide. In some aspects, the at least one heterologous nucleic acid sequence further encodes an adjuvant. In further embodiments, the adjuvant is an AB5 toxin.

In some aspects, the disclosure is drawn to a recombinant hookworm cell comprising a polynucleotide comprising one or more control sequences operably linked to at least one heterologous nucleic acid sequence, wherein the at least on heterologous nucleic acid sequence encodes a vaccine antigen and/or a therapeutic polypeptide.

In some aspects, the disclosure is drawn to a pharmaceutical composition comprising a pharmaceutically acceptable carrier, and a transgenic helminth comprising cells containing a polynucleotide comprising one or more control sequences operably linked to at least one heterologous nucleic acid sequences, wherein the at least one heterologous nucleic acid sequences encodes a vaccine antigen and/or a therapeutic polypeptide. In a further aspect, the transgenic helminth is a hookworm.

In some aspects, the transgenic helminth comprises a polynucleotide sequence comprising SEQ ID NO:1 and/or 2. In some aspects, the transgenic helminth comprises one or more polynucleotide sequences sharing at least 90% sequence identity with SEQ ID NO:1 and/or 2.

In some aspects, the transgenic helminth comprises a polynucleotide sequence encoding one or more polypeptide sequences comprising SEQ ID NO:3, 4 and/or 5. In some aspects, the transgenic helminth comprises a polynucleotide sequence encoding one or more polypeptide sequences that share at least 90% sequence identity with SEQ ID NO: 3, 4, and/or 5.

### BRIEF DESCRIPTION OF THE FIGURES

FIG. 1 depict a micrograph of a cross-section image of an adult hookworm attached to a gut wall.

FIG. 2 depicts the life cycle of the Necator americanus hookworm as the infestation begins when an infective larva (L3) penetrates the skin and migrates to the small intestine via the circulatory system and the lungs. The L1 will undergo two molts to the L3, which is infective for the host.

FIG. 3 depicts vector pLVX containing the hookworm g-tubulin promoter driving the eGFP gene.

FIG. 4 depicts the process of microinjected lentivirus into the sperm sac of 16-day-old males (P0).

FIG. 5 depicts the transgenic F1 L3 and wt L3 hookworms imaged by spectral confocal microscopy. Worm 1 was visible by dissecting scope, while worm 2 was detected only under the confocal microscope. The left panels of Worm 1 and Worm 2 feature two different emission wavelength profiles that corresponding to the region at the center of each of the crosses on the worms in the panels to the right of Worm 1 and Worm 2. The solid/dashed lines clearly identify the region corresponding to the distinct emission wavelength profiles.

FIG. 6 depicts emission fingerprinting mode on Zeiss 710 spectral confocal microscope, which was used to differentiate the eGFP and autofluorescence signals by linear un-mixing. Worm 1 and Worm 2 panels identify transformed and untransformed hookworms.

FIG. 7 depicts a schematic of one of many contemplated vector constructs for delivery of broadly neutralizing antibody VRC01 by a helminth, and in one instance a hookworm. The pLVX vector is engineered to express the VRC01 heavy chain and light chain as a single polypeptide. Processing of the 2A self-cleaving peptide sequence liberates the two chains, which then form a functional antibody. Expression is driven in this example by the hookworm ASP-5 promoter sequence.

### DETAILED DESCRIPTION

### Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, in particular embodiments, the terms “about” or “approximately” when preceding a numerical value indicates the value plus or minus a range of 10%.

As used herein, “isolate,” “isolated,” “isolated helminth,” and like terms, are intended to mean that the one or more helminth has been separated from at least one of the materials with which it is associated in a particular environment (for example soil, water, animal tissue).

Thus, an “isolated helminth” does not exist in its naturally occurring environment; rather, it is through the various techniques described herein that the helminth has been removed from its natural setting and placed into a non-naturally occurring state of existence. Thus, the isolated variant or isolated helminth may exist as, for example, a biologically pure sample (or other forms of the isolate) in association with an acceptable carrier.

As used herein, “helminth” is intended to mean organisms recognized under phyla Platyhelminths, Nematoda, and Acanthocephala.

As used herein, “endoparasitic nematode” is intended to mean a nematode that has at least one life stage, e.g., larvae, that lives in the internal organs or tissues of a host.

As used herein, “hookworm” is intended to mean organisms recognized under genera Ancyclostoma, and Necator.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration, such as a carrier utilized for dermal administration. The choice of carrier can be selected with regard to standard pharmaceutical practice.

The terms “control sequence” or “control sequences” as used herein are to be interpreted as sequences that control transcriptional and/or translational initiation, elongation, or termination. Control sequences include: promoters, enhancers, operators, repressors, terminators, secretion signal, etc.

The term “growth medium” as used herein, is any medium which is suitable to support growth of a microbe or an organism of the present disclosure. By way of example, the media may be natural or artificial including gastrin supplemental agar, LB media, blood serum, and tissue culture gels. It should be appreciated that the media may be used alone or in combination with one or more other media. It may also be used with or without the addition of exogenous nutrients.

The medium may be amended or enriched with additional compounds or components, for example, a component which may assist in the interaction and/or selection of specific groups of microorganisms. For example, antibiotics (such as penicillin) or sterilants (for example, quaternary ammonium salts and oxidizing agents) could be present and/or the physical conditions (such as salinity, nutrients (for example organic and inorganic minerals (such as phosphorus, nitrogenous salts, ammonia, potassium and micronutrients such as cobalt and magnesium), pH, and/or temperature) could be amended.

A “recombination” or “recombination event” as used herein refers to a chromosomal crossing over or independent assortment. The term “recombinant” refers to an organism having a new genetic makeup arising as a result of a recombination event.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions among when compared against one another. Mapping of molecular markers in the vicinity of an allele is a procedure which can be performed by the average person skilled in molecular-biological techniques.

As used herein, the term “genotype” refers to the genetic makeup of an individual cell, cell culture, tissue, organism, or group of organisms.

As used herein, the term “phenotype” refers to the observable characteristics of an individual cell, cell culture, organism (e.g., a mammal), or group of organisms which results from the interaction between that individual's genetic makeup (i.e., genotype) and the environment.

As used herein, the term “chimeric” or “recombinant” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “mammal” refers to humans, dogs, cats, goats, sheep, primates, non-human primates, cows, horses, rodents, rabbit, hares, bats, canines, foxes, wolves, raccoons, bears, deer, antelope, buffalo, ermine, mink, chinchilla, and other animals known to be mammals.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458. For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (1989) Molecular Cloning: A Laboratory Manual (2nd ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 500% of a complementary target sequence hybridizes to a perfectly matched probe or primer. Typically, stringent conditions will be those in which the salt concentration is less than about 1.0 M Na+ion, typically about 0.01 to 1.0 M Na+ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1 SSC at 60° C. Hybridization procedures are well known in the art and are described by e.g. Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 160/%, 17%, 18%, 19% or 20%, followed by a wash in 5×SSC, containing 0.1% (w/v) sodium dodecyl sulfate, at 55° C. to 65° C.

As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, a “constitutive promoter” is a promoter which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues or at certain life stages/cycles.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other.

For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature). In some embodiments, a recombinant construct(s) include CRISPR-cas9 elements that facilitate the genetic manipulation of the cells/organisms of the present disclosure by inserting one or more genes of interest into the cells/organisms.

In some embodiments, the cell or organism has at least one heterologous trait. As used herein, the term “heterologous trait” refers to a phenotype imparted to a transformed host cell or transgenic organism by an exogenous DNA segment, heterologous polynucleotide or heterologous nucleic acid. Various changes in phenotype are of interest to the present disclosure, including but not limited to expression of antibodies, expression of heterologous proteins, and the like. These results can be achieved by providing expression of heterologous products or increased expression of endogenous products in organisms using the methods and compositions of the present disclosure.

Helminths

In some embodiments, the present disclosure provides isolated helminths belonging to groups known as trematodes, cestodes, and nematodes.

In some embodiments, the present disclosure provides isolated cestodes belonging to the group commonly known as tapeworms, including those belonging to the following genera: Hymenolepis, Echinococcus, Taenia, and Diphyllobothrium.

In some embodiments, the present disclosure provides isolated nematodes belonging to the following genera: Necator, Ancylostoma, Agrioslomum, Bunostomum, Cyclodontostomum, Galonchus, Monodonttus, Uncinaria, Enterobius, Trichuris, Capillostrongyloides, Liniscus, Orthomninx, Pearsonema, Sclerotrichum, Strongyloides, and Tenoranema.

In some embodiments, the present disclosure provides isolated trematodes belonging to the following genera: Schistosoma, Nanophyetus, Alaria, Heterobilharzia, Heterophyes, Metagonimus, Cryptocotyle, Apophallus, Opisthorchis, Platynosonium, Metorchis, and Eurytremna.

In some embodiments, the present disclosure provides isolated hookworms (phylum Nematoda) belonging to the following species: Ancylostoma braziliense, Ancylostoma caninum, Ancylostoma ceylanicum, Ancylostoma duodenale, Ancylostoma pluridentatum, Ancylostoma tubaeforme, Necator americanus, and Uncinaria stenocephala.

In some embodiments, the present disclosure provides endoparasitic nematodes. In some embodiments, the helminths are obtained, at various locales, from the circulatory system or gastrointestinal tract of animals in the United States. In some embodiments, the helminths are an established laboratory line.

In some embodiments, the delivery of vaccine antigens by helminths will allow uptake of antigens at the mucosal surface, generating specific immune responses that cannot be easily generated by parenteral routes of administration.

In some embodiments, the use of helminths to delivery biological therapies will allow the delivery of proteins and other molecules to the gut without exposure to the harsh denaturing environment of the stomach, thereby retaining activity of the proteins and other molecules.

In some embodiments, engineered helminths will continuously secrete the therapeutic agent(s) at the gut mucosa and into the bloodstream over their lifespan in the host, obviating the need for multiple treatments. In some embodiments, the treatment(s) could be discontinued at any time by administering an antihelmintic agent.

A. Hookworms

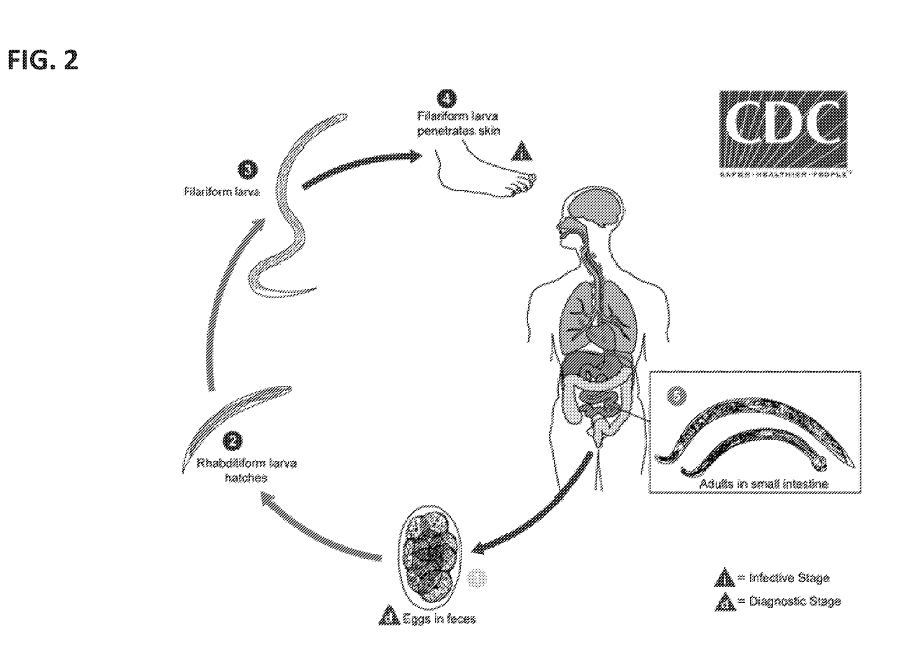

Hookworm infection remains one of the greatest public health threats worldwide, with an estimated 800 million people infected. Hookworm infection begins when an infective larva (L3) penetrates the skin and migrates to the small intestine via the circulatory system and the lungs (FIG. 2). Heavy hookworm infection is the leading cause of anemia in the tropics, resulting in debilitating and sometimes fatal iron-deficiency anemia caused by blood loss to feeding adult worms in the intestine. Children, pregnant women, and the elderly are particularly susceptible to morbidity from hookworm infection. Control strategies are restricted to periodic de-worming of infected individuals, which is limited by rapid re-infection rates and the development of drug resistant worm populations. Vaccine efforts suffer for the lack of effective target antigens. Development of new drug targets and improved vaccine antigens will require a better understanding of hookworm biology, particularly the infective process. However, the obligate requirement for a host and the inability to grow the complete life cycle in vitro precluded development of powerful genetic tools for hookworm research until recently. The present disclosure is drawn to using other parasitic helminths to develop transient transfection methods for helminths (hookworms) that will provide a foundation for the future development of heritable transformation technology.

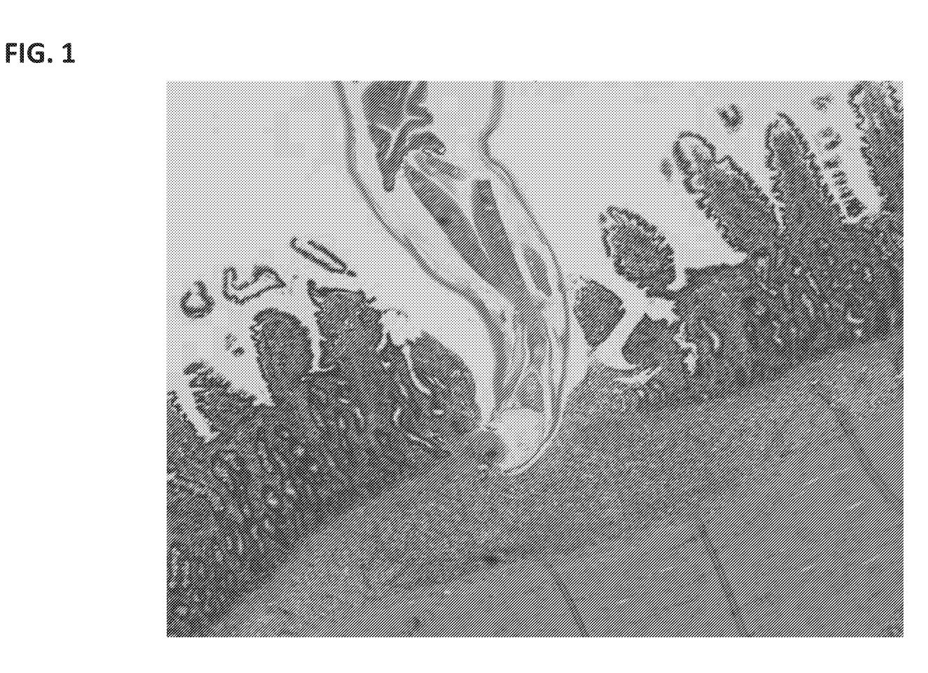

The present disclosure includes making and using helminths as a vector system that may be engineered to express biological molecules such as proteins in mammals. The helminths may be engineered to express a biological payload in the circulatory system, gastrointestinal tract, and/or mucosal surface. As a vector system, helminths, such as hookworms, have several advantages. Unlike viral vectors, hookworms can be removed after the desired effect is achieved, e.g., protective immune response. Because they live as adults attached to the small intestine feeding on blood, they can deliver the biological agent at the mucosal surface and into the circulatory system (FIG. 1). Hookworms also undergo a migration in the circulatory system, and could be engineered to express their payload once in the bloodstream.

Further modification of helminths could direct biological payloads to the gut without having to first be exposed to the harsh denaturing environment of the stomach, thereby retaining biological activity and avoiding the need for parenteral administration. In some aspects, transformed helminths, e.g., hookworms, are selected for optimal expression of the biological payloads and ultimately transferred to the host orally or percutaneously where the hookworms may pass through the circulatory system and ultimately mature in the lower gastrointestinal tract. The delivery of therapeutics may be controlled through genetic mechanisms such as hookworm stage-specific promoters that are active only in certain tissues, or through anthelmintics that are capable of safely clearing all hookworms from the host.

Helminth Engineering

Helminths such as hookworms are engineered to become a delivery system for biological molecules of interest, allowing for the continuous or discontinuous delivery of the molecules in the circulatory system and the gut mucosa. In some embodiments, particle bombardment is contemplated for introducing reporter genes into helminth embryos and larval stages, and transformants are assayed for survival, growth, and reported gene expression.

In some embodiments, antibody selection techniques described by Giordano-Santini et al. (2010. Nat. Methods. 7:721-723) and Semple et al. (2010. Nat. Methods. 7:725-727) are utilized to select transformants from non-transformants. In one embodiment, a nematode transformation vector carrying a neomycin resistance gene can be utilized to confer neomycin resistance to G-418 on both wt C. elegans and C. briggsae. The system allows for hands-off maintenance and enrichmot of transgenic worms carrying non-integrated transgenes on selective plants. The marker can also be used for Mosl-mediated single-copy insertion in wild-type genetic backgrounds. See Giodano-Santini et al. In one embodiment, a puromycin selection system allows for the rapid and easy isolation of large populations of transgenic nematodes, which further allows for the selection of single-copy transgenes and does not require any specific genetic background. See Semple et al.

In some embodiments, chemical and lipid-based technology is utilized to introduce reporter genes, and other gene of interest, into molting helminth (hookworm) larvae.

In some embodiments, the introduction of foreign or heterologous polynucleotides is performed with a piggybac retrotransposon-based integrating vector for introducing transgenes into helminth chromosomes. The development of helminth (hookworm) transfection protocol represents a significant advance for research, and allows for the determination of helminth expression and function in a homologous genetic context for the first time. Furthermore, transgenesis now allows for the development of novel helminth control strategies.

In some embodiments, the following helminth promoters are utilized in the vectors: asp-1, asp-2, asp-3, asp-4, asp-5, snr-3, lpp-1, tbg-1, myo-2, myo-3, ges-1, eft-3, ama-1, daf-11, daf-16, daf-21, daf-2, let-858, unc-119, vit-2, sur-5, hlh-13, pie-1, and spe-11. In some embodiments, characterized promoter sequences are utilized as comparison sequences for isolating orthologous genes from hookworms and other helminths, for use in the vectors of the present disclosure. Praitis et al. (2011. Methods in Cell Bio. Academic Press. 106:159-185); Evans (2006. Transformation and Microinjection: WormBook).

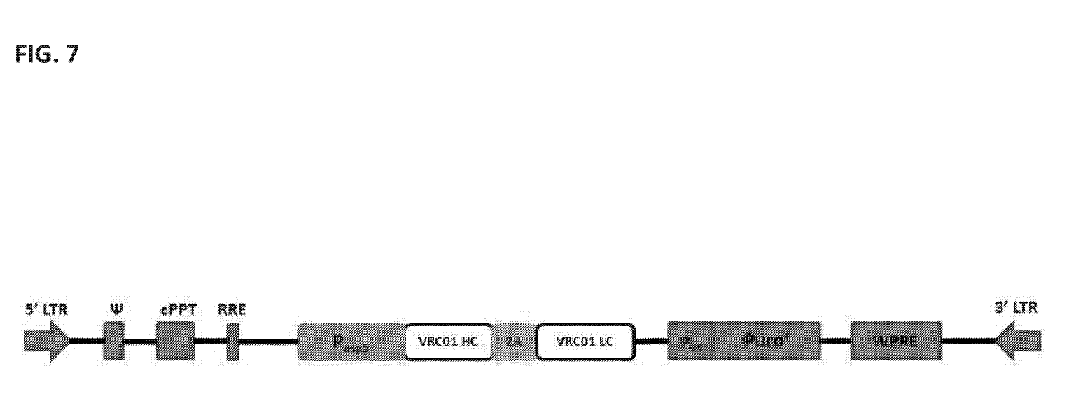

In one embodiment, the exemplary vector shown in FIG. 7 is utilized to transform one or more helminths to deliver a biological payload of choice. In some embodiments, the VRC01 sequences may be substituted with other sequences/genes of the present disclosure for delivery to a mammalian host. In some embodiments, the sequences/genes of the present disclosure contemplated for insertion into one or more helminths are heterologous to the one or more helminths.

Helminth Compositions

In some embodiments, the helminth compositions of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium sulfate; magnesium sulfate; magnesium oxide; and products of vegetable origin.

In some embodiments, the helminth compositions of the present disclosure are liquid. Where liquid embodiments are used, it may be desired to include one or more carrier materials including, but not limited to: a solvent that may include water or an alcohol, and other food-grade solvents. In some embodiments, the helminth compositions of the present disclosure include binders such as polymers, carboxymethylcellulose, starch, polyvinyl alcohol, and the like.

In some embodiments, microbial compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts and combinations thereof. In a further embodiment, helminth compositions comprise polymers of agar, agarose, gelrite, gellan gum, and the like. In some embodiments, helminth compositions comprise plastic capsules, emulsions (e.g., water and oil), membranes, and artificial membranes. In some embodiments, emulsions or linked polymer solutions may comprise microbial compositions of the present disclosure. See Harel and Bennett (U.S. Pat. No. 8,460,726B2).

In some embodiments, the helminth composition of the present disclosure comprises a food, beverage, paste, cream, and the like.

In some embodiments, the helminth compositions of the present disclosure comprise helminth eggs, rhabditiform larva, filariform larva, or adults. In some embodiments, the helminth compositions of the present disclosure comprise a combination of one or more of the following: helminth eggs, rhabditiform larva, filariform larva, parasitic 3^{rd} larval stage, parasitic 4^{th} larval stage, or adults.

In some embodiments, the helminth compositions of the present disclosure comprise two or more helminths of different species or variants. In some embodiments, the two or more helminths of different species or variants may be in the form of an egg, larva, and/or adult.

In some embodiments, the helminth compositions of the present disclosure comprise adjuvants, which may be selected from the following: flagellin, Escherichia coli heat labile toxin, cholera toxin, AB5 toxin, a viral coat protein, a chemokine, a cytokine, and a defensin.

Administration of Helminth Compositions

In some embodiments, the helminth compositions of the present disclosure are administered to mammals, including humans. In some embodiments, helminth compositions of the present disclosure are administered via the oral route in the form of a drink, food, or pill. In some embodiments, the helminth compositions of the present disclosure are administered via a dermal application in which the helminth composition is applied directly onto the skin at the foot, leg, arm, hand, neck, or chest.

In some embodiments, the helminth composition is administered in a dose comprise a total of, or at least, 0.2 ml, 0.4 ml, 0.6 ml, 0.8 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 m, 42 ml, 43 ml, 44 ml, 45 ml, 46 ml, 47 ml, 48 ml, 49 ml, 50 ml, 60 ml, 70 ml, 80 ml, 90 ml, 100 ml, 200 ml, 300 ml, 400 ml, 500 ml, 600 ml, 700 ml, 800 ml, 900 ml, or 1,000 ml.

In some embodiments, the helminth composition is administered in a dose comprising a total of, or at least, 10⁹, 10⁸, 10⁷, 10⁶, 10⁵, 10⁴, 10³, 10², or 10 helminths.

In some embodiments, the helminth compositions are administered in a dose comprising 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹ 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total helminths.

In some embodiments, the helminth composition is administered 1 or more times per day. In some aspects, the composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per day.

In some embodiments, the helminth composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per week.

In some embodiments, the helminth composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some embodiments, the helminth composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

In some embodiments, the helminth composition may be used in crude form and need not be isolated from an animal or a media. For example, tissues, feces, or growth media which includes the helminths For example, fresh feces could be obtained and optionally processed

In some embodiments, the administration of helminths of the present disclosure results in the helminths remaining present in the host for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 days.

In some embodiments, the administration of helminths of the present disclosure results in the helminths remaining present in the host for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 weeks.

In some embodiments, the administration of helminths of the present disclosure results in the helminths remaining present in the host for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, 10, 11, or 12 months.

In some embodiments, the administration of helminths of the present disclosure results in the helminths remaining present in the host for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, 10, 11, or 12 years.

In some embodiments, is desirable to clear the administered helminths from the host, and which time antihelminthics are administered, resulting in a 100% clearance of the helminths from the host.

Molecules for Biodelivery

In some embodiments, biological treatments are delivered to the gut by without exposure to the harsh denaturing environment of the stomach, thereby retaining their activity and avoiding the need for parenteral administration. Engineered helminths (hookworms) may secrete their payload continuously for several years, obviating the need for multiple treatments.

In some aspects, the present disclosure is drawn to administering one or more helminth compositions as a vector for the delivery of biomolecules in a host. In some embodiments, the biomolecules include the following non-limiting biologics (antigens, therapeutics, etc.): antigens, insulin, antibacterial molecules, antimicrobial molecules, viricidal molecules, bactericidal molecules, lysozyme, insulin, recombinant insulin, myoglobin, calcitonin, interleukin, recombinant human interleukin-4, granulocyte-macrophage colony-stimulating factor (GM-CSF), DAS181, interferon, interferon gamma, interleukin-2, sargramostin, alpha-1 antitrypsin, vasoactive intestinal peptide, glutathione, human growth hormone, interferon beta, interferon alpha, human parathyroid hormone, recombinant methionyl human granulocyte colony-stimulating factor (r-huG-CSF), PEGylated rhG-CSF, erythropoietin, EPO-Fc, heparin, antibodies, antibody-like molecules, antibody fragments, diabodies, hepatitis B surface antigen, diphtheria toxin, bovine serum albumin, follicle-stimulating hormone, prolactin, thyroid-stimulating hormone, vasopressin, vasopressin-analogue, Factor IX, immunoconjugates, protein C, albumin, calcitonin, leuprolide, cetrorelix, PYY (3-36), glucagon, glucagon-like peptide-1 (GLP-1), oxytocin, detirelix, renin-inhibitory peptides, cyclosporine, cyclosporin A, RGD peptide, vasoactive intestinal peptide (VIP), vaccines, and adjuvants.

In some aspects, the biomolecule is an antibody. In further aspects, the antibodies are neutralizing antibodies. In further aspects, the antibodies are HIV neutralizing antibodies. In further aspects, the antibodies target the CD4 binding site of the HIV envelope. In further aspects, the antibodies are VRC01 antibodies.

In some embodiments, the vector encoding the biomolecule comprises the VRCOI heavy chain (SEQ ID NO: 1). In some embodiments, the vector encoding the biomolecule comprises the VRC01 kappa chain (SEQ ID NO:2). In some embodiments, the vector encodes the ADF47181.1 anti-HIV immunoglobulin heavy chain variable region (SEQ ID NO:3). In some embodiments, the vector encodes the ADF47184.1 anti-HIV immunoglobulin light chain variable region (SEQ ID NO:4). In some embodiments, the vector encodes the FMDV 2A peptide (SEQ ID NO:5). In some embodiments, the FMDV 2A vector comprises a cleavage site. In further embodiments, the FMDV 2A vector comprises a cleavage site between the proline and glycine residues of SEQ ID NO:5.

In some embodiments, the biomolecules of the present disclosure are encoded by a polynucleotide sequence sharing at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs:1 or 2.

In some embodiments, the biomolecules of the present disclosure share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs:3, 4, or 5.

Beneficial Phenotypic Outcomes

In some embodiments, the present disclosure is drawn to administering one or more helminth compositions to mammals to treat an infection, disease, disorder, inflammation. In some embodiments, the administering of one or more helminth compositions to mammals is to immunize, inoculate, or vaccinate the mammals. In some embodiments, the administering of one or more helminth compositions to mammals is for disease, disorder, or syndrome prophylaxis.

In some embodiments, the administration of one or more helminth composition to mammals is for the treatment or prevention of viral infection, bacterial infection, fungal infection, and nematode infection.

In some embodiments, the administration of one or more helminth composition to mammals is for the treatment or prevention of rheumatoid arthritis, lupus, celiac disease, Sjogren's syndrome, polymyalgia rheuatica, multiple sclerosis, ankylosing spondylitis, type-1 diabetes, alopecia areata, vasculitis, temporal arteritis, Grave's disease, inflammatory bowel disease, psoriasis, systemic lupus erythematosus, autoimmune thyroiditis, Goodpasture's disease, alopecia areata, antiphospholipid antibody syndrome, Guillain-Barre syndrome, Hashimoto's disease, hemolytic anemia, idiopathic thrombocytopenic purpura, inflammatory myopathies, myasthenia gravis, primary biliary cirrhosis, scleroderma, and vitiligo.

In some embodiments, the administration of one or more helminth compositions to mammals is for the treatment or prevention of cancer, human papilloma virus, influenza, measles, mumps, rubella, polio, coxsachie, rotavirus, rabies, hepatitis A, hepatitis B, HIV, HTLV-1, cytomegalovirus, herpes 1, herpes 2, yellow fever, dengue fever, West Nile virus, lassa, hanta virus, Ebola, and Marburg

HIV

Despite advances in antiretroviral drug treatments and combination prevention modalities, HIV infection is still a major cause of mortality and morbidity. Around 37 million people are infected worldwide, and access to antiretroviral drugs is not guaranteed. Emphasis has shifted from prophalaxis to a “cure.” Persisetence of the latent reservoir is considered the major obstacle to the eradication of HIV. Approaches such as “shock and kill” whereby the latent virus is reactivated and intensification of anti-retroviral therapy (ART) is provided has not yet led to a cure given that the virus returns when ART is withdrawn. In order to drive down virus replication further, a potent immune response would be of enourmous benefit to help contain the virus as it reemerged. The considerable obstacles to an effective HIV vaccine are known by skilled practitioners to be great. It is widely believed that an effective vaccine should induce both humoral and cell mediated immunity. However, antibodies can provide a potent defense against HIV, and induction of such activity is considered a cornerstone of a successful vaccine (Haynes et al. 2014). Indeed, in the follow up studies for the RV144 HIV vaccine trial, one of the correlates of vaccine immunity was an antibody response against the V2 region of HIV envelope (Yates et al. 2014). Follow up trials have been designed to recapitulate the immunity induced, but developing an immunogen to elicit broad neutralizing antibodies has been elusive. The immunity induced must be prominent at mucosal surfaces where the majority of HIV infection occurs. Therefore, methods that deliver antigen at a mucosal surface will be expected to generate the desired humoral and cell mediated immunity.

The present disclosure sets forth methods of utilizing bioengineered helminths, e.g., hookworms, to deliver HIV vaccine antigens and/or neutralizing antibodies, including bNAbs to the intestinal mucosa to prevent and/or treat HIV infection. In some embodiments, helminth vectors allow antigen delivery directly to the intestinal mucosa, continuous antigen boosting until the desired immune response is generated and easy removal when the appropriate response is achieved.

Hookworms live in the small intestine where they attach to the mucosa and feed on blood. This places them in an ideal location to deliver the desired molecular payload to both the mucosal surface and the systemic circulation. The inventors of the invention(s) disclosed herein are believed to be the first group to effectively and repeatedly engineer hookworms to express vaccine antigens and/or therapeutic neutralizing antibodies.

When present in high numbers in the small intestine, hookworms can cause anemia due to their ability to attach to the mucosa and suck blood. However, low worm burdens of N. americanus are safe and potentially beneficial, as demonstrated by several recent clinical trials (Croese et al. 2013; Croese et al. 2006; Feary et al. 2009, 39:1060-68; Mortimer et al. 2006; Daveson et al. 2011). This mutualism provides a unique opportunity to exploit the intimate relationship between the feeding hookworm and the small intestine mucosa for therapy. Engineered hookworms could continuously secrete a therapeutic agent, such as a vaccine antigen or an active biological, at the gut mucosa and into the bloodstream. This alternate route of administration provides several advantages over current therapeutic methods. First, biological treatments could be delivered to the gut without exposure to the harsh denaturing environment of the stomach, thereby retaining their activity and avoiding the need for parenteral administration. Second, hookworms would secrete their payload continuously for up to several years, obviating the need for multiple treatments. Third, unlike viral vectors, the treatment could be discontinued at any time by administering an anthelmintic.

The present disclosure contemplates transgenic engineered hookworms to deliver the HIV envelope V2 region to the mucosal surface of the small intestine in host animals. Studies will include monitoring and characterization of the antibody and cell mediated immune response generated by this exposure. In some aspects, transgenic hookworms are administered to humans, in which the hookworms develop in the small intestine and secrete the desired molecule at the mucosal surface. Direct delivery of vaccine antigens to the intestinal mucosa is expected to generate a prominent immune response at mucosal surfaces where the majority of HIV infection occurs, and blood feeding by the worm will introduce the antigen into circulation, generating a systemic immunity also.

Neutralizing antibodies have been used to successfully treat SIV infections, but require continuous administration. Viral vectors can be used, but are undesirable because they persist in the host and cannot be removed. Using the hookworm delivery system, anti-HIV neutralizing antibodies would be expressed continuously at the mucosal surface to suppress or prevent HIV infection (West et al. 2012). In addition to mucosal and systemic introduction of their payload, transgenic hookworms can be easily removed by drug treatment when the desired effect is reached (e.g. protective immune response), a significant advantage over viral vectors, and would be able to continuously secreted neutralizing antibodies for a period of several years. Low doses of the human hookworm Necator americanus have been used safely in human clinical trials as “helminth therapy” to treat several diseases, and have been approved by the FDA for administration to humans for the development of a hookworm vaccine challenge model.

Recently, new antibodies have been generated which neutralize different viral envelopes in vitro (West A P, Jr., et al: Structural insights on the role of antibodies in HIV-1 vaccine and therapy. Cell 2014, 156:633-648). One such antibody is VRC01, which targets the CD4 binding site of the HIV envelope (Zhou T, et al: Structural basis for broad and potent neutralization of HIV-1 by antibody VRC01. Science 2010, 329:811-817). In addition to their role in vaccine induced immunity, passive transfers of anti-HIV/SIV antibodies have prevented establishment of infection in a primate model, and delayed rebound of plasma virus after treatment cessation in humans (Ruprecht R M: Passive Immunization with Human Neutralizing Monoclonal Antibodies Against HIV-1 in Macaque Models: Experimental Approaches. In Therapeutic Antibodies. Volume 525. Edited by Dimitrov A S: Humana Press; 2009: 559-566: Methods in Molecular Biology™; rkola A, et al: Delay of HIV-1 rebound after cessation of antiretroviral therapy through passive transfer of human neutralizing antibodies. Nature Medicine 2005, 11:615-622).

Passive therapy is also being considered as part of immune support in “shock and kill” strategies for HIV latent cell eradication. The use of broadly neutralizing antibodies (bNAbs) could be used as part of therapeutic strategies to PREVENT, as well as TREAT, HIV infection. Frequent administration of antibodies is an impediment to the implementation of such therapies. One successful approach to deliver antibodies used an adeno-associated virus (AAV) vector expressing antibody-like, SIV-specific immunoadhesins (Johnson P R, et al: Vector-mediated gene transfer engenders long-lived neutralizing activity and protection against SIV infection in monkeys. Nat Med 2009, 15:901-906). In the animal model, they generated long-lasting neutralizing activity in serum and completely protected against challenge with virulent SIV. However, AAV is unlikely to gain acceptance for a vector approach in humans since it persists within the host. Hookworm vectors would be able to safely deliver engineered immunoadhesions to the intestinal mucosa and systemically for extended periods (up to 7 years) from a single infection, and can quickly and easily be removed when needed. We will deliver neutralizing antibodies that target the CD4 binding site of the HIV envelope to the small intestine mucosa of host animals using engineered hookworms. Our prototype antibody will be VRC01, which we will deliver as single-chain fragment variable immunoadhesins (scFv) expressing both the variable heavy and light chains of anti-HIV NAs. We will monitor their appearance of fully folded functional antibody in the systemic circulation and at the intestinal mucosa.

### EXAMPLES

### Example I: General Methods

Parasites:

We use the laboratory model hookworm Ancylostoma ceylanicum to develop the engineering technology. A. ceylanicum is primarily a parasite of dogs and cats, but also infects a significant number of people in Southeast Asia (Traub 2013). Importantly, it can complete its life cycle in hamsters, which allows easy manipulation and isolation of parasitic adult stages.

Vector Constructs:

We use two integrating vectors to transform hookworms. The retrotransposon piggyBac integrates into many genomes including the related parasitic nematode Strongyloides stercoralis and the trematode Schistosoma mansoni (Handler 2002; Perera et al. 2002; Ding et al. 2005; Wilson et al. 2007; Balu et al. 2005; Shao et al. 2012; and Morales et al. 2007). We also use non-replicating, integrating retro- and/or lentivirus vectors pseudotyped with the VSV-G envelop protein to broaden their host range. The pseudotyped retroviral vector MLV has been used successfully to transform S. mansoni (Rinaldi et al. 2012; and Mann et al. 2014). Both vectors integrate into target genomes, resulting in heritable germline transformation.

Transformation:

We have developed a particle ballistics-based protocol from transfection of hookworms that has shown early success. The piggyBac constructs together with helper transposase mRNA (to facilitate integration) is co-precipitated onto gold particles. Young adult stage A. ceylanicum worms recovered from infected hamsters are bombarded with the coated gold particles using a BioRad PDS-1000/He Particle Bombardment System. Following recovery, the bombarded adults are transferred to uninfected hamsters by oral gavage (15-20 bombarded females: 5-7 non-bombarded males per hamster). A proportion of these worms survive and reproduce, as determined by the presence of F1 eggs in their feces. These eggs are cultured to the infective L3 and are utilized to infect hamsters. When these F1 adults begin reproducing, they are recovered from hamsters and allowed to lay eggs in vitro for 1-2 days. The adult worms are screened by PCR to determine if they are transfected. If positive, their F2 eggs are raised to L3 and used to establish the transfected line by infecting hamsters. Once each transgenic line is established, it is further evaluated to confirm that the insert is integrated and producing the recombinant protein. Transgenes are delivered by using retro- or lentiviral vectors by exposing free-living larval stages (egg, L1, L2) to virions containing the constructs. Once the worms reach the infective L3 stage they are used to infect hamsters, and transgenic lines established as described above.

Vaccine Delivery:

To deliver an HIV antigen using hookworm, we insert the cDNA encoding the V2 region of HIV envelope in plasmid pXL-BacII downstream of the Ace-asp-5 promoter sequence. Ace-asp-5 encodes a secreted CAP domain protein of unknown function (Siwinska et al. 2013). Expression of this gene is up-regulated more than 100-fold in adult hookworms. Therefore, the promoter drives expression of the V2 region in the appropriate tissue in the hookworm, resulting in its active secretion specifically by the adult stage when it is attached to the mucosa. In some embodiments, the same promoter is used to drive expression of the V2 cDNA in pseudotyped retro- or lentiviral vectors (e.g. pLNHX and pLenti6.2 V5-DEST).

Confirmation of antigen expression—adult V2 transgenic hookworms are recovered from infected hamsters and cultured in vitro to collect secreted products. The products are examined by Western blot and ELISA to confirm secretion of the V2.

Generation of an immune response—monitor infected hamsters for the generation of a systemic and mucosal anti-V2 antibody and/or cell mediated immune response.

Neutralizing Antibody (NA) Delivery:

Utilize same promoter and vectors described in vaccine delivery to express chimeric antibody-like immunoadhesins with HIV specificity. The variable heavy and variable light chains from VRC01 Fab molecular clones previously shown to neutralize HIV are joined by a linker and then attached to a human IgG Fc fragment as described. The resulting immunoadhesin is inserted into pXL-BacII or a viral vector for transfection of hookworms.

Confirm that the immunoadhesion neutralizes HIV—in vitro synthesized NA is tested for binding to CD4 binding site of the HIV-1 envelope.

Generation of the NA transgenic hookworm line-ballistics/virus, confirmation by PCR

Confirmation of NA expression—adult transgenic hookworms expressing the immunoadhesins are recovered from infected hamsters and cultured in vitro to collect secreted products. The products are examined by ELISA to confirm NA secretion.

Confirmation of secreted antibodies in the mucosal fluids and systemic circulation of infected hamsters.

### Example II: Generation of Transgenic Hookworms

Anthropophilic species of hookworm (Necator americanus, Ancylostoma duodenale, Ancylostoma ceylanicum) are transfected with a DNA construct designed to secrete the desired molecule under the control of a hookworm promoter. Depending on the desired effect, stage specific promoters could be employed to express the molecule only in the infective migrating stage, the adult stage, or throughout the parasitic life cycle.

The desired cDNA is inserted downstream of a hookworm specific promoter. For secretion of the molecule in larval stages, the promoter from the hookworm asp-1 gene is used. ASP-1 is synthesized and secreted only during the infective L3 stage. Therefore, this promoter is only active in that stage, and hence the payload only produced and secreted then. For secretion during by the hookworm adult, the promoter for the gene encoding ASP-5 will be used. ASP-5 is secreted by the adult stage only, so once the worm matures, it continuously secretes the target protein for its remaining life.

Constructs are made in the vector pXL-BacII derived from the piggyBac transposon. This vector integrates into the hookworm genome in the presence of helper transposase, provided either as a second plasmid or as mRNA, resulting in a heritable transformation of the worm. The vector may also be engineered to contain a marker, either a fluorescent protein gene (e.g. GFP) or an antibiotic resistance gene (e.g. Neo) to facilitate selection of transgenic worms.

Creating Transgenic Hookworm Lines Using Biolistic Bombardment

Preparing Machine and Consumables for Bombardment:
Machine setting of BioRad PDS-1000/He particle delivery system for hookworm adults
   Set gap distance between rupture disk retaining cap and microcarrier launch assembly to ¼ inch
   3 cm target shelf
   2200 psi rupture disk
   27-28 inches Hg of vacuum
Autoclave the following items prior to use:
   Microcarrier holder
   Rupture disk cap
   Launch assembly

Preparing Gold Beads Stock:
Weigh out 15 mg of 1 um gold beads into a 1.5 ml siliconized Eppendorf tube.
Add 1 ml of 70% ethanol (v/v). Vortex vigorously for 5-10 minutes.
Allow particle to settle for 15 mins.
Spin briefly for 3-5 seconds in a benchtop microfuge, remove supernatant.
Add 1 ml autoclaved distilled water, vortex 1 min; allow particles to settle for 1 min; spin briefly in a microfuge; remove supernatant.
Repeat step 5 for 2 more times.
Add 250 ul sterile 50% glycerol to bring the gold beads concentration to 60 mg/ml. Store at 4° C.

Coating DNA and/or RNA with Gold Beads:
The amounts given below are for one bombardment, using BioRad PDS-1000/He particle delivery system.
Vortex gold beads stock for at least 5 mins.
Aliquot 20 ul of stock beads into a 1.5 ml siliconized Eppendorf tube, using siliconized micropipette tips, vortex for at least 5 mins.
Add in the following order and vortex 1 mins between each addition.
   Add DNA and RNA mixture up to 80 ul (4 ul 2 ug/ul piggybac helper mRNA+piggybac vectors, including A.cey ama-1p::gfp in pXL-BacII. A.cey ama-1p::NLS gfp in pXL-BacII)
   20 ul 2.5M CaCl2
   10 ul 0.1M spermidine (kept @4° C.)
Vortex for at least 3 mins. Allow beads to settle for 1 min.
Spin briefly for 3-5 seconds in a benchtop microfuge, remove supernatant.
Resuspend in 60 ul 70% ETOH (v/v) and vortex 1 min.
Spin briefly for 3-5 seconds in a benchtop microfuge, remove supernatant.
Resuspend in 15 ul 100% ETOH, vortex until ready to load onto the microcarrier.

Loading Gold Beads Solution onto Microcarriers
Sterilize the microcarrier in 100% ETOH and allow to dry in drying chamber (glass petri dish with calcium carbonate, and set microcarrier on a piece of filter paper, add lid).
Once microcarrier is dry, use the red insertion tool to place it onto the microcarrier holder, which shall be autoclaved beforehand.
Pipet beads solution to break up any clumping. Spread solution over the center region of the microcarrier, let it dry in drying chamber. The beads outside of the center hole in holder is not going to be shoot towards worms, so load beads twice to make sure beads only spread in the center.

Performing a Bombardment
Prepare hookworms for bombardment
Sacrifice 5 hamsters for each bombardment, which are infected by 80 L3s 12-13 days before. We usually can get 100-120 young adults out of 5 hamsters. Leave adults in RPMI-C with or without filtered serum in a small petri dish on 37° C. hotplate. Hand pick worms to new dishes several times to get rid of the gut tissue. Separate 25-30 males out of the entire worm population. Right before bombardment, transfer all worms excluding 25-30 males onto a prewarmed 10 cm NGM plate. Push worms into the center of the plate using a small transfer pipet. Keep lid open until worms are just dry on the hotplate. It's critical that worms are dry otherwise worms maybe burst out of the plate.

Bombard Worms
Place everything in the bombardment chamber
Briefly wet the 2200 psi rupture disk in isopropanol using a sterile forceps. Load the disk into recess of retaining cap while it's still wet.
Screw the rupture disk retaining cap onto the gas acceleration tube, tighten up with a torque wrench.
Assemble the microcarrier launch by orderly placing sterile stopping screen (dip in 100% ETOH and flame to sterilize), microcarrier holder with microcarrier, cover lid.
Install the microcarrier launch assembly in the top slot inside the bombardment chamber.
Place the just dried worm plate on the target shelf in the second slot.
Close chamber door.
Fire the machine
Turn on power button on the PDS-1000/He.
Turn on vacuum source, set vacuum switch to VAC position until proper vacuum level achieved.
Press vacuum switch to HOLD position to hold vacuum level. Turn off vacuum source.
Turn on Helium tank.
Press and hold FIRE button to allow pressure build up in the gas acceleration tube until the rupture disk burst, which will be indicated by a loud pop sound.
Set the VACUUM switch to VENT position to release vacuum in the chamber.
Remove consumables out of chamber and shut down machine and helium tank.

Post-Bombardment Care of Hookworms
Leave bombarded worm petri dish on hotplate for several mins.
Wash worms into RPMI-C in a small petri dish on 37° C. hotplate. Let them recover for 1 hour.
Infect 4-6 hamsters with 20-25 live bombarded females and 5 non-bombard males for each hamster.
F1 will show up in feces after 7-10 days of reinfection.

Creating Transgenic Hookworm Lines Using Lentivirus Microinjection

Lentivirus Production and Concentration

Using the Lenti-X HTX Packaging Systems (Clontech) to Produce Lentiviral Supernatants
Approximately 24 hr before transfection, seed Lenti-X 293T cells in T75 flask, in 10 ml of growth medium. Make sure that the cells are plated evenly. Incubate at 37° C., 5% CO2 overnight. Continue to incubate the cells until you are ready to add the transfection mixture in Step 7. The cells should be 80-90% confluent at the time of transfection.
Thoroughly vortex Xfect Polymer.
For each transfection sample, prepare two microcentrifuge tubes by adding reagents in the order listed:
   Tube 1 (Plasmid DNA)
      557 μl Xfect Reaction Buffer
      36 μl Lenti-X HTX Packaging Mix 7 μl Lenti-X Vector DNA (1 μg/μl)
      600 μl Total Volume
   Tube 2 (Polymer)
      592.5 μl Xfect Reaction Buffer
      7.5 μl Xfect Polymer 600 μl Total Volume
Vortex each tube well to mix.
Add the Polymer solution (Tube 2) to the DNA solution (Tube 1) and vortex well at a medium speed for 10 sec.
Incubate each DNA-Xfect mixture for 10 min at room temperature to allow nanoparticle complexes to form.
Add the entire 1200 μl of DNA-Xfect solution from Step 5 dropwise to cultured cells from Step 1. Rock the plate gently back and forth to mix.
Incubate the plate at 37° C.
After 6-8 hours, replace the transfection medium with 10 ml fresh complete growth medium (containing Tet System Approved FBS) and incubate at 37° C. for an additional 24-48 hr. Viral titers will generally be highest at 48 hr after the start of transfection. Caution: discarded medium contains infectious lentivirus.
48 hr after the transfection, harvest the lentiviral supernatants and pool similar stocks, if desired. Centrifuge briefly (500×g for 10 min) to remove cellulardebris.
Concentrate the virus at 50,000×g for 90 mins at 4° C. Remove the supernatant, resuspend the virus pellet in 0.5-1% of the original volume in PBS on ice.
Verify virus production with Lenti-X GoStix then use the fresh virus to transduce hookworm sperm, and store the leftover virus at −80° C. (snap freeze).

Lentivirus Titration
Immediately before microinjection of virus into the seminal vesicle of hookworms, we use Instant Lentivirus Test Clontech's Lenti-X GoStix to assess the quality of the virus by detect lentiviral p24.
Take 20 μl of your lentiviral supernatant and apply to the sample well (S) of the GoStix cassette.
Add 4 drops of Chase Buffer to the sample well and wait for the bands to develop.
A control band will always appear (C), and a test band (T) will start to appear within 30-180 sec and reach maximum intensity at 10 mins if your sample contains sufficient lentivirus. A clear Test band will estimate the virus titer to be >5×105 IFU/ml.

Lentivirus Microinjection
Prepare hookworms for microinjection
Sacrifice hamsters for microinjection, which are infected by 100 L3s A. ceylanicum 16 days before. Leave A. ceylanicum adults in RPMI-C in a small petri dish on 37° C. hotplate. Hand pick worms to new dishes several times to get rid of the gut tissue.

Pick males and females into separate dishes, and keep females in 37° C. incubator with 5% CO2, while injecting males.

Microinject Virus into Male Seminal Vesicle
Aliquot approximately 1 ul of concentrated virus at the end of the injection needle. Wait until the liquid fills the tip of needle.
Place a drop of halocarbon oil onto a microinjection pad, then place a piece of capillary needle in the center of the oil. Use a dissecting microscope to visualize.
Fix the injection needle to the holder of micromanipulator and position the needle tip in the center of the oil and pointed towards the piece of capillary needle.
Rub the needle against capillary so the tip of the needle breaks at an angle. Check if fluid flows through the needle properly.
Transfer several male hookworms onto microinjection pad under dissecting microscope. First, transfer worms onto NGM plate, then use a C. elegans regular worm picker to transfer worms onto injection pad.
Identify the location of the seminal vesicle (where spermatids are stored before ejaculation) by its darker and round shape in the middle of worm body, then cover only the seminal vesicle with halocarbon oil for injection.
Place the injection slide with worms on gliding stage of microinjection system, and move the worm towards the injection needle until the worm cuticle is penetrated and needle tip is inside the seminalvesicle.
Apply pressure to inject virus solution into worm until liquid flow is noticeable.
Recover worms by picking from injection pad into 37° C. RPMI-C with 20% filtered dog serum.
Culture injected males at 37° C. with 5% CO2 until all males are injected.

Hookworm Reinfection and F1 Transgene Screen
Transfer injected male P0 and non-injected females into naive hamsters to mate. Progeny (F1) will show up in the feces 7-10 days after transfer. Examine progeny for transgene (EGFP) expression by fluorescent microscopy in the F1 generation. Infect naïve hamsters with L3 expressing the transgene to establish a transgenic line.

Administration of Transgenic Hookworms

Hookworms are Administered to Hosts:
Infective L3 stage of the transgenic hookworm line is maintained in healthy, virus-free donor animals or humans.
Feces from infected donors are collected and cultured with activated charcoal to raise infective L3 hookworms according to standard methods.
Infective L3 hookworms are recovered from culture by standard methods, and disinfected by multiple washes in sterile buffer containing antibiotics.
Patients are infected by application of 10-30 L3 hookworms to the skin using a gauze pad.
The infection site is monitored for 1 hour.
Weekly stool samples are taken beginning at approximately 4 weeks post-infection to monitor for the presence of eggs, which indicates that the worms have matured.
Blood samples are taken to monitor for evidence of protein secretion, as well as to monitor for development of anemia.

### Example III: In Vivo Infection of Hookworm Sperm with Lentivirus

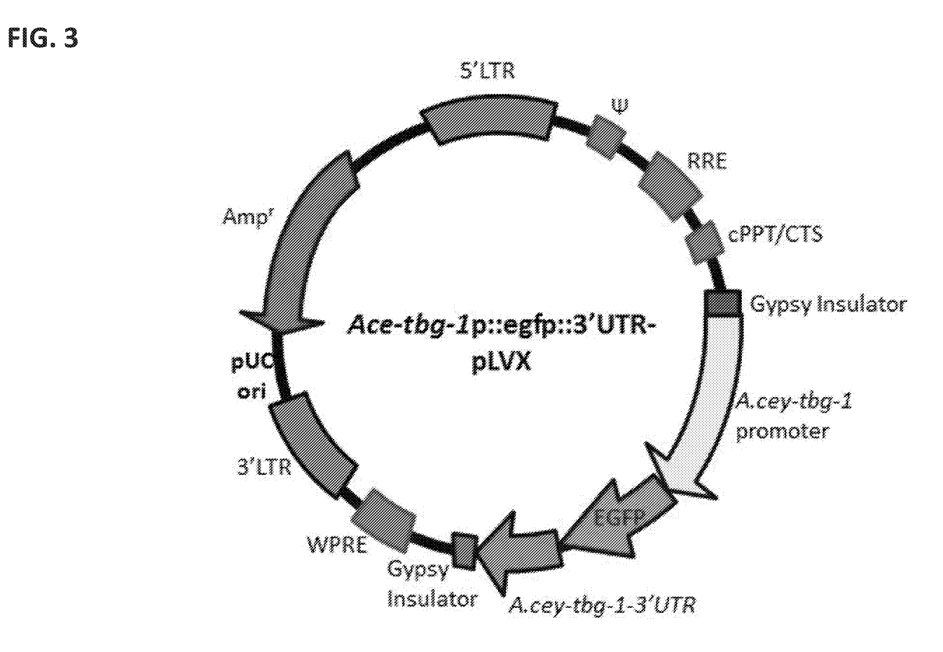

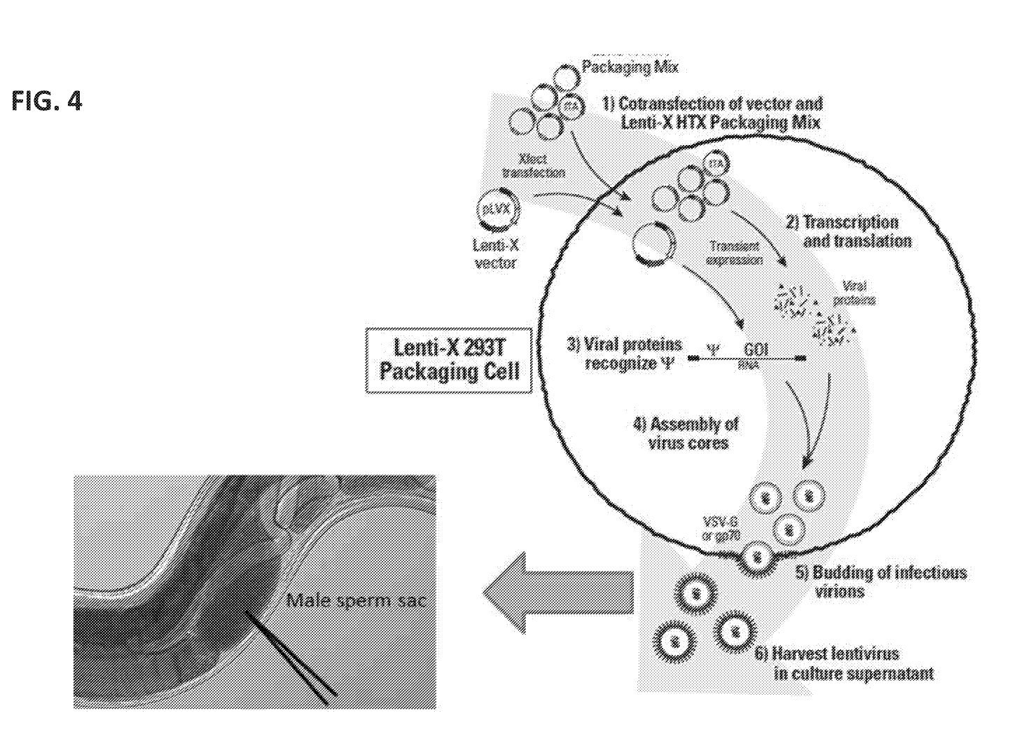

A transfected Lentivirus vector (Ace-tbg-1p::egfp::3′UTR-pLVX) was created and mixed along with Lenti-X HTX packaging mix into Lenti-X 293 T cells (FIG. 3 and FIG. 4). The resulting lentivirus were harvested and concentrated by ultracentrifugation of the T cell composition 48 hours post infection. The concentrated lentivirus were titrated using Lenti-X GoStix to 5×10⁵ IFU/ml.

Adult hookworms were obtained from donor hamsters, and the sperm sac of 16 day adult males (P0) hookworm males were microinjected with the concentrated lentivirus. The injected males were transferred with an equal number of females into naïve hamsters. The resulting cultured F1 L3 stage hookworms were examined under a Zeiss 710 spectral confocal microscope under lambda mode using a 488 nm laser. The emission signals were captured in 10 nm bands.

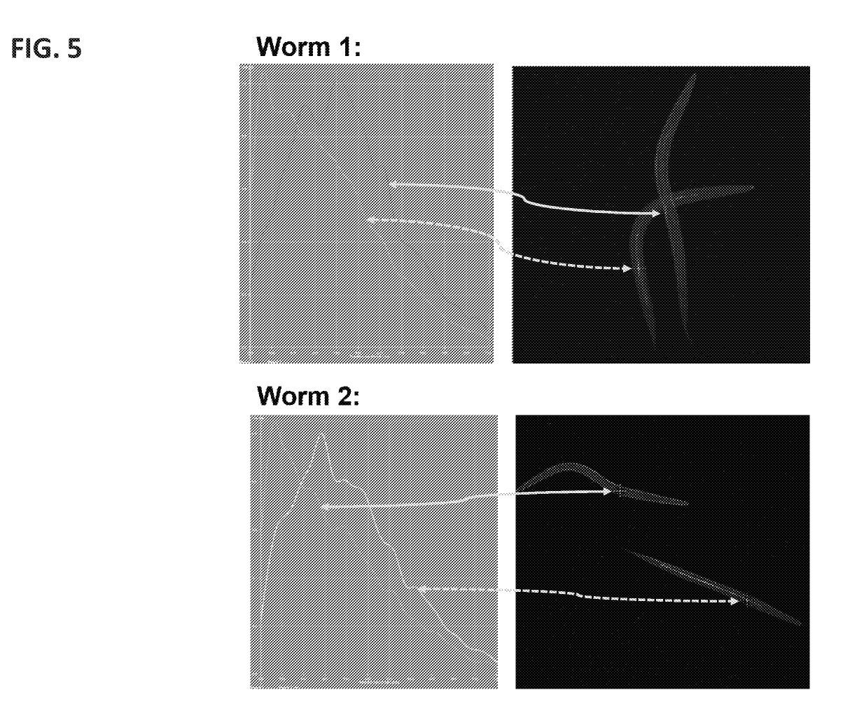

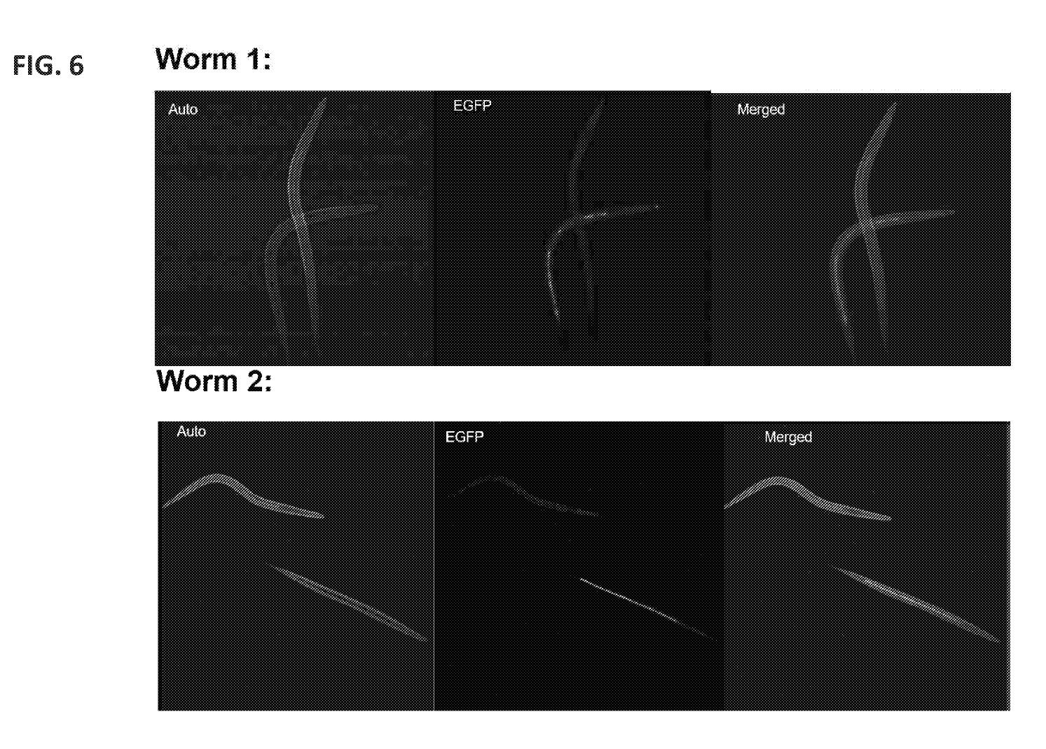

Each of FIG. 5 and FIG. 6 comprise fluorescent micrographs of wild type and transgenic F1 L3 stage hookworms. FIG. 5 is divided into two sets of hookworms, “Worm 1” and “Worm 2.” Due to autofluorescence, both worms fluoresce, however the transgenic worm can be distinguished from the wild type worm because it exhibits the intense amount of fluorescence along the middle of the worm. FIG. 6 is also divided into two sets of hookworms, “Worm 1” and “Worm 2.” FIG. 6 differs from FIG. 5 in that FIG. 6 utilized the emission fingerprinting mode to subtract out the autofluorescence signal from the fluorescent panels labelled “EGFP,” clearly identifying the transgenic hookworm as the only worm fluorescing.

Each of FIG. 1 and FIG. 2 comprise fluorescent micrographs of wild type and transgenic F1 L3 stage hookworms. FIG. 5 is divided into two sets of hookworms, “Worm 1” and “Worm 2.” Due to autofluorescence, both worms fluoresce, however the transgenic worm can be distinguished from the wild type worm because it exhibits the intense amount of fluorescence along the middle of the worm.

This example clearly identifies that hookworms were successfully transformed to express a heterologous polypeptide.

### REFERENCES

1. West et al. J. Virol. 2012, 86:195-202.
2. Croese et al. Int. J. Parasitol. 2013. 43:275-282.
3. Croese et al. Gut. 2006. 55:136-137.
4. Feary et al. Clin. Exp. Allergy. 2009. 39:1060-1068.
5. Feary et al. Clin. Exp. Allergy. 2009. 40:299-306.
6. Mortimer et al. Am. J. Trop. Med. Hyg. 2006. 75:914-920.
7. Beyrer et al. Curr. Opin. HIV AIDS. 2013. 8:306-310.
8. Cohen et al. Lancet. 2013. 382:1515-1524.
9. Daveson et al. PLoS One. 2011. 6:e17366.
10. Haynes et al. J. Allergy Clin. Immuno. 2014. 134:3-10.
11. Yates et al. Sci. Transl. Med. 2014. 6:228ra239.
12. West et al. Cell. 2014. 156:633-648.
13. Zhou et al. Science. 2010. 329:811-817.
14. Ruprecht R M. In Therapeutic Antibodies. Volume 525. Edited by Dimitrov A S: Humana Press. 2009:559-566.
15. Trkola et al. Nature Medicine. 2005. 11:615-622.
16. Johnson et al. Nat. Med. 2009. 15:901-906.
17. Traub R J. Int. J. Parasitol. 2013. 43:1009-1015.
18. Handler A M. Insect Biochemistry and Molecular Biology. 2002. 32:1211-1220.
19. Perera et al. Insect Mol. Biol. 2002. 11:291-297.
20. Ding et al. Cell. 2005. 122:473-483.
21. Wilson et al. Mol. Ther. 2007. 15:139-145.
22. Balu et al. PNAS. 2005. 102:16391-16396.
23. Shao et al. PLoS Pathog. 2012. 8:e1002871.
24. Morales et al. FASEB J. 2007. 21:3479-3489.
25. Rinaldi et al. PLoS Pathog. 2012. 8:e1002820.
26. Mann et al. Transgenic Res. 2014. 23:539-556.
27. Siwinska et al. Acta Parasitologica. 2013. 58:112-118.
28. Boyle et al. Int. J. Parasitol. 2003. 33:1259-1268.
29. Xu et al. Int. J. Parasitol. 2011. 41:355-362.
30. Kalinna et al. TRENDS Parasitol. 23(5): 197-204.
31. Osada et al. J. Biomed. Biotechnol. 2010. Article ID 743758:1-9
32. Johnston et al. Parasitol. 2009. 136:125-147.
33. Yoo et al. Nat. Rev. Drug Discovery. 10:521-535.
34. Whelan et al. Mol. Therapy. 2014. 22(10):1730-1740.
35. Rashtak et al. Aliment Pharmacol. Ther. 2012. 35:768-781.
36. Wu et al. Science. 2010. 329:856-861.
37. Claesen et al. Synth. Biol. 2014. Special Issue (Natural Products):A-G.
38. U.S. Pre-grant Patent Application Publication 2002/0090363 A1.
39. Giordano-Santini et al. 2010. Nat. Methods. 7:721-723.
40. Semple et al. 2010. Nat. Methods. 7:725-727.
41. Balazs et al. 2011. Nature. 481(7379):81-84.
42. Chinnasamy et al. 2006. Virology. 3:14.
43. Torres et al. 2010. J. of Biotechnology. 146(3):138-142.
44. Verrier et al. 2011. PLoS ONE. 6(5):e20553.

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment of any form of suggestion that they constitute valid prior art or form part of the common general knowledge in any country in the world.

What is claimed is:

1. A transgenic helminth comprising: cells containing a polynucleotide sequence comprising one or more control sequences operably linked to at least one heterologous nucleic acid sequence, wherein the at least one heterologous nucleic acid sequence encodes a vaccine antigen and/or a therapeutic polypeptide.

2. The transgenic helminth of claim 1, wherein the helminth is a trematode, cestode, or nematode.

3. The transgenic helminth of claim 2, wherein the helminth is a nematode selected from the genera consisting of: Necator, Ancylostoma, Agriostomum, Bunostomum, Cyclodontostomum, Galonchus, Aonodontus, Uncinaria, Enterobius, Trichuris, Capillostrongyloides, Liniscus, Orthominx, Pearsonema, Sclerotrichum, Strongyloides, and Tenoranema.

4. The transgenic helminth of claim 1, wherein the transgenic helminth is a transgenic hookworm.

5. The transgenic helminth of claim 3, wherein the nematode is a hookworm selected from species of: Ancylostoma braziliense, Ancylostoma caninum, Ancylostoma ceylanicum, Ancylostoma duodenale, Ancylostoma pluridentatum, Ancylostoma tubaeforme, Necator americanus, and Uncinaria stenocephala.

6. The transgenic hookworm of claim 4, wherein the vaccine antigen is an HIV polypeptide.

7. The transgenic hookworm of claim 6, wherein the HIV polypeptide is an envelope V2 region polypeptide.

8. The transgenic hookworm of claim 4, wherein the therapeutic polypeptide is selected from the group consisting of: insulin, gamma-interferon, beta-interferon, Factor VIII, Factor IX, tissue plasminogen activator, human growth hormone, bovine growth hormone, and a neutralizing antibody.

9. The transgenic hookworm of claim 8, wherein the therapeutic polypeptide is aneutralizing antibody, which neutralizes viral envelope proteins.

10. The transgenic hookworm of claim 9, wherein the neutralizing antibody neutralizes HIV envelope proteins.

11. The transgenic hookworm of claim 8, wherein the therapeutic polypeptide is a neutralizing antibody of VRC01.

12. The transgenic hookworm of claim 4, wherein the at least one heterologous nucleic acid sequence further encodes an adjuvant.

13. The transgenic hookworm of claim 12, wherein the adjuvant is selected from the group consisting of: flagellin, Escherichia coli heat labile toxin, cholera toxin, an AB5 toxin, a viral coat protein, a chemokine, a cytokine, and a defensin.

14. The transgenic hookworm of claim 4, wherein the one or more control sequences is selected from the group consisting of: a promoter, a terminator, a secretion signal, an enhancer, and an operator.

15. The transgenic hookworm of claim 14, wherein the one or more control sequences is a hookworm promoter.

16. The transgenic hookworm of claim 15, wherein the hookworm promoter is selected from asp-1, asp-2, asp-3, asp-4, asp-5, snr-3, lpp-1, tbg-1, myo-2, myo-3, ges-1, eft-3, ama-1, daf-11, daf-16, daf-21, daf-2, let-858, unc-119, vit-2, sur-5, hlh-13, pie-1, and spe-11.

17. The transgenic hookworm of claim 15, wherein the hookworm promoter is a stage-specific promoter that is parasitic L3 stage-specific, L4 stage-specific, or adult stage-specific.

18. The transgenic hookworm of claim 4, wherein the at least one heterologous nucleic acid sequence encodes an HIV envelope V2 region and an AB5 toxin.

19. A method of preparing a transgenic helminth, the method comprising introducing into cells of a helminth a polynucleotide comprising one or more control sequences operably linked to at least one heterologous nucleic acid sequence,
wherein the at least one heterologous nucleic acid sequence encodes a vaccine antigen and/or a therapeutic polypeptide.

20. The method of claim 19, wherein the helminth is a trematode, cestode, or nematode.

21. The method of claim 20, wherein the helminth is a nematode selected from the genera consisting of: Necator, Ancylostoma, Agriostomum, Bunostonmum, Cyckdontostomum, Galonchus, Monodonttus, Uncinaria, Enterobius, Trichuris, Capillostrongyloides, Liniscus, Orthominx, Pearsonema, Sclerotrichum, Sirongyloides, and Tenoranema.

22. The method of claim 19, wherein the transgenic helminth is a transgenic hookworm.

23. The method of claim 22, wherein the introducing a polynucleotide into cells of the helminth comprises biolistic bombardment or viral transfection.

24. The method of claim 22, wherein the vaccine antigen is an HIV polypeptide.

25. The method of claim 24, wherein the HIV polypeptide is an envelope V2 region polypeptide.

26. The method of claim 19, wherein the therapeutic polypeptide is selected from the group consisting of: insulin, gamma-interferon, beta-interferon, Factor VIII, Factor IX, tissue plasminogen activator, human growth hormone, bovine growth hormone, and a neutralizing antibody.

27. The method of claim 26, wherein the therapeutic polypeptide is a neutralizing antibody, which neutralizes viral envelope proteins.

28. The method of claim 27, wherein the neutralizing antibody neutralizes HIV envelope proteins.

29. The method of claim 26, wherein the therapeutic polypeptide is a neutralizing antibody of VRC01.

30. The method of claim 25, wherein the hookworm is selected from the group consisting of: Ancylostoma braziliense, Ancylostoma caninum, Ancylostoma ceylanicum, Ancylostoma duodenale, Ancylostoma pluridentatum, Ancylostoma tubaeforme, Necator americanus, and Uncinaria stenocephala.

31. The method of claim 25, wherein the at least one heterologous nucleic acid sequence further encodes an adjuvant.

32. The method of claim 31, wherein the adjuvant is selected from the group consisting of: flagellin, Escherichia coli heat labile toxin, cholera toxin, an AB5 toxin, a viral coat protein, a chemokine, a cytokine, and a defensin.

33. The method of claim 25, wherein the one or more control sequences is selected from the group consisting of: a promoter, a terminator, a secretion signal, an enhancer, and an operator.

34. The method of claim 33, wherein the one or more control sequences is a hookworm promoter.

35. The method of claim 34, wherein the hookworm promoter is a stage-specific promoter that is parasitic L3 stage-specific, L4 stage-specific, or adult stage-specific.

36. The method of claim 34, wherein the hookworm promoter is selected from asp-1, asp-2, asp-3, asp-4, asp-5, snr-3, lpp-1, tbg-1, myo-2, myo-3, ges-1, eft-3, ama-1, daf-11, daf-16, daf-21, daf-2, let-858, unc-119, vit-2, sur-5, hlh-13, pie-1, and spe-11.

37. The method of claim 25, wherein the at least one heterologous nucleic acid sequence encodes an HIV envelope V2 region and an AB5 toxin.

38. The method of claim 25, wherein the method of introducing a polynucleotide into cells of a hookworm comprises viral transfection utilizing a lentivirus vector to introduce the polynucleotide, wherein the lentivirus vector has been pseudotyped with a VSV-G envelope protein.

39. The method of claim 25, wherein the method of introducing a polynucleotide into cells of a hookworm comprises viral transfection utilizing a retrovirus vector to introduce the polynucleotide, wherein the retrovirus vector has been pseudotyped with a VSV-G envelope protein.

40. A method of delivering one or more polypeptides to a mammalian circulatory system or intestinal tract, the method comprising:
delivering, via either an oral or percutaneous route, a composition comprising a transgenic helminth comprising cells containing a polynucleotide comprising one or more control sequences operably linked to at least one heterologous nucleic acid sequence,
wherein the at least one heterologous nucleic acid sequence encodes a vaccine antigen and/or a therapeutic polypeptide.

41. The method of claim 40, wherein the helminth is a trematode, cestode, or nematode.

42. The method of claim 41, wherein the helminth is a nematode selected from the genera consisting of: Necator, Ancylostoma, Agriostomrum, Bunostomum, Cyclodontostomum, Galonchus, Monodontus, Uncinaria, Enterobius, Trichuris, Capillostrongyloides, Liniscus, Orthominx, Pearsonema, Sclerotrichum, Strongyloides, and Tenoranema.

43. The method of claim 40, wherein the transgenic helminth is a transgenic hookworm.

44. The method of claim 43, wherein the vaccine antigen is an HIV polypeptide.

45. The method of claim 44, wherein the HIV polypeptide is an envelope V2 region.

46. The method of claim 43, wherein the therapeutic polypeptide is selected from the group consisting of: insulin, gamma-interferon, beta-interferon, Factor VIII, Factor IX, tissue plasminogen activator, human growth hormone, bovine growth hormone, and a neutralizing antibody.

47. The method of claim 46, wherein the therapeutic polypeptide is a neutralizing antibody, which neutralizes viral envelope proteins.

48. The method of claim 47, wherein the neutralizing antibody neutralizes HIV envelope proteins.

49. The method of claim 46, wherein the therapeutic polypeptide is a neutralizing antibody of VRC01.

50. The method of claim 43, wherein the at least one heterologous nucleic acid sequence further encodes an adjuvant.

51. The method of claim 50, wherein the adjuvant is selected from the group consisting of: flagellin, Escherichia coli heat labile toxin, cholera toxin, an AB5 toxin, a viral coat protein, a chemokine, a cytokine, and a defensin.

52. The method of claim 43, wherein the one or more control sequences may be selected from the group consisting of: a promoter, a terminator, a secretion signal, an enhancer, and an operator.

53. The method of claim 52, wherein the one or more control sequences is a hookworm promoter.

54. The method of claim 53, wherein the hookworm promoter is a stage-specific promoter that is parasitic L3 stage-specific, L4 stage-specific, or adult stage-specific.

55. The method of claim 43, wherein the at least one heterologous nucleic acid sequence encodes an HIV envelope V2 region and an AB5 toxin.

56. A method of treating an HIV-infected patient, the method comprising: orally or percutaneously administering the transgenic hookworm of claim 10 to a patient in need thereof.

57. The method of claim 56, wherein about one to three months post-hookworm introduction, the patient exhibits a decreased HIV titer, as compared to the HIV titer immediately before hookworm introduction.

58. The method of claim 56, wherein the neutralizing antibody is VRCOI1.

59. A method of HIV prophylaxis in a patient, the method comprising orally or percutaneously administering the transgenic hookworm of claim 4 to a patient in need thereof.

60. The method of claim 59, wherein the patient is protected from HIV infection at a greater occurrence than a patient not having been administered the transgenic hookworm.

61. The method of claim 59, wherein the HIV polypeptide is an envelope V2 region polypeptide.

62. The method of claim 61, wherein the at least one heterologous nucleic acid sequence further encodes an adjuvant.

63. The method of claim 62, wherein the adjuvant is an AB5 toxin.

64. A recombinant hookworm cell, comprising: a polynucleotide comprising one or more control sequences operably linked to at least one heterologous nucleic acid sequence,
wherein the at least one heterologous nucleic acid sequence encodes a vaccine antigen and/or a therapeutic polypeptide.

65. A pharmaceutical composition, comprising:
a) a transgenic helminth comprising cells containing a polynucleotide comprising one or more control sequences operably linked to at least one heterologous nucleic acid sequence,
wherein the at least one heterologous nucleic acid sequence encodes a vaccine antigen and/or a therapeutic polypeptide; and
b) a pharmaceutically acceptable carrier.

66. The pharmaceutical composition of claim 65, wherein the transgenic helminth is a hookworm.

67. The transgenic helminth of claim 4, wherein the polynucleotide sequence comprises SEQ ID NO:1 and/or SEQ ID NO:2.

68. The transgenic helminth of claim 67, wherein the polynucleotide sequence comprises one or more sequences that share at least 90% sequence identity with SEQ ID NO: I1 and/or SEQ ID NO:2.

69. The transgenic helminth of claim 4, wherein the polynucleotide sequence encodes one or more polypeptide sequences comprising SEQ ID NO:3, 4, and/or 5.

70. The transgenic helminth of claim 69, wherein the polynucleotide sequence encodes one or more polypeptide sequences that share at least 90% sequence identity with SEQ ID NO:3, 4, and/or 5.


SEQUENCE LISTING (5 sequences)

SEQ ID NO: 1 - DNA, 363 bp, Artificial Sequence
  Polynucleotide sequence encoding VRC01 heavy chain
caggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgactcgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatca

SEQ ID NO: 2 - DNA, 309 bp, Artificial Sequence
  Polynucleotide sequence encoding VRC01 kappa chain
gaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaag

SEQ ID NO: 3 - protein, 121 aa, Homo sapiens
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 4 - protein, 104 aa, Homo sapiens
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 5 - protein, 21 aa, Artificial Sequence
  FMDV 2A peptide sequence
VKQTLNFDLLKLAGDVESNPG